(19) United States
(12) Patent Application Publication
(10) Pub. No.: US 2022/0074906 A1
(43) Pub. Date: Mar. 10, 2022

(54) DEVICE AND METHOD FOR MEASURING RADON RELEASE AMOUNT DURING ROCK SHEARING DAMAGE PROCESS

(71) Applicant: Institute of Rock and Soil Mechanics, Chinese Academy of Sciences, Wuhan City (CN)

(72) Inventors: Chuanqing ZHANG, Wuhan City (CN); Lingyu LI, Wuhan City (CN); Guojian CUI, Wuhan City (CN); Yang GAO, Wuhan City (CN); Hui ZHOU, Wuhan City (CN); Dawei HU, Wuhan City (CN); Jingjing LU, Wuhan City (CN)

(21) Appl. No.: 17/373,796

(22) Filed: Jul. 13, 2021

(30) Foreign Application Priority Data
Sep. 7, 2020 (CN) 202010925972.7

(51) Int. Cl. G01N 33/00; G01N 1/22; G01N 3/24

(52) U.S. Cl. CPC G01N 33/0055; G01N 2203/0658; G01N 3/24; G01N 1/2226

(57) ABSTRACT

The disclosure provides a device and method for measuring radon release amount during rock shearing damage process. The method includes: the sealed chamber where the rock sample placed is vacuumed in the first place, and then the radon released during rock sample shearing damage process is all collected into the radon collection box, and then the concentration of the radon collected in the radon collection box is measured with a radon concentration measure instrument, so that the purity of the radon collected in the radon collection box can be ensured, and thus the accuracy of the concentration of radon measured by the radon concentration measure instrument can be ensured, and the device and method have good practicability.

### TECHNICAL FIELD

The disclosure relates to the technical field of geotechnical engineering, and in particular to a device and method for measuring radon release amount during rock shearing damage process.

### BACKGROUND OF THE INVENTION

As the underground engineering advances into the depths of the earth, deep underground engineering is facing an increasingly serious environmental safety problem, that is, the hazard of toxic gas-radon. Radon is ubiquitous in geological bodies. The release and migration of radon are closely related to the degree of rupture of the geological body. Rock rupture will increase the amount of radon being released, and after rupture, a connected migration channel will be formed, which is conducive to the rapid migration of free radon to a cavern space. The accumulation of radon to a certain concentration will have a serious impact on the health of the experimenters and construction personnel in the cavern. Therefore, it is particularly important to study the correlation between the rupture of the surrounding rock and the radon concentration in the underground cavern.

In the existing technology, a method for measuring the radon concentration of the rock is to use nitrogen as a carrier gas to drive out the radon released during the damage of a rock sample from the rock sample. The device used in this method is complicated and the radon is mixed with nitrogen gas, so that the measurement of the volume concentration of radon will be affected by nitrogen gas, resulting in inaccurate measurement results.

### SUMMARY OF THE INVENTION

In view of the defects of the existing technology, the disclosure provides a device and method for measuring radon release amount during rock shearing damage process, so as to improve the accuracy of the test results.

The technical solutions of the disclosure is as below.

In one aspect, the disclosure provides a device for measuring radon release amount during rock shearing damage process, the device comprising:

a shear experiment instrument, the shear experiment instrument including a normal loading mechanism, a tangential loading mechanism, a sealed chamber and a shear box for placing a rock sample, the shear box being disposed in the sealed chamber, the normal loading mechanism and the tangential loading mechanism being disposed on the outside of the sealed chamber, and an output end of the normal loading mechanism being movable back and forth along a vertical direction, the output end of the normal loading mechanism being capable of passing through the sealed chamber and the output end of the normal loading mechanism acting on a top of the shear box, and an output end of the tangential loading mechanism being movable back and forth along a horizontal direction, the output end of the tangential loading mechanism being capable of passing through a horizontal side of the sealed chamber, and the output end of the tangential loading mechanism acting on the horizontal side of the shear box;

an acoustic emission sensor and an acoustic emission processor, a plurality of the acoustic emission sensors being disposed on a surface of the rock sample at intervals, and a plurality of the acoustic emission sensors being connected to the acoustic emission processor;

a vacuum pump, the vacuum pump being communicated with an interior of the sealed chamber;

a radon collection box, the radon collection box being communicated with the interior of the sealed chamber.

a radon concentration measure instrument, the radon concentration measure instrument being communicated with the radon collection box.

In some embodiments, a top of the sealed chamber is arranged with an opening, and the top of the sealed chamber is provided with a detachable cover plate, and the opening is sealed by the cover plate.

In some embodiments, the cover plate is formed with a first through hole, and a first sealing gasket is fixedly arranged on an outer side of the cover plate, and the first through hole is covered by the first sealing gasket; and

the normal loading mechanism includes a normal cylinder and a normal loading head, the normal cylinder being fixedly disposed right above the cover plate, a telescopic end of the normal cylinder being extendable and retractable downward in a vertical direction, an end of the normal loading head being fixedly connected to the telescopic end of the normal cylinder, and an other end of the normal loading head sequentially passing through the middle of the first sealing gasket and the first through hole in the middle of the cover plate, and an other end of the normal loading head acting on the top of the shear box.

In some embodiments, the horizontal side of the sealed chamber is formed with a second through hole, and a second sealing gasket is fixedly provided on the outer side of the sealed chamber, and the second through hole is covered by the second sealing gasket; and

the tangential loading mechanism includes a tangential cylinder and a tangential loading head; the tangential cylinder is fixedly disposed on an outer side of the horizontal side of the sealed chamber, a telescopic end of the tangential cylinder being extendable and retractable in a horizontal direction, an end of the tangential loading head being fixedly connected to the telescopic end of the tangential cylinder, an other end of the tangential loading head sequentially passing through the middle of the second sealing gasket and the second through hole on the horizontal side of the sealed chamber, and an other end of the tangential loading head acting on a horizontal side of the shear box.

Furthermore, the shear box includes a first box body and a second box body, and the first box body is arranged above the second box body; the opposing end faces of the first box body and the second box body are both arranged with an opening, an end of the first box body away from the tangential loading mechanism is fixedly provided with a counterforce frame; and the bottom of the second box body is arranged on a roller bead row.

In some embodiments, a plurality of the acoustic emission sensors are arranged on a surface of the shear sample by a coupling agent.

In some embodiments, the device includes a first three-way valve, a second three-way valve and a third three-way valve, wherein:

the sealed chamber being provided with a vent hole, the vent hole being communicated to a first port of the first three-way valve through an air outlet pipeline, and a second port of the first three-way valve being communicated to an end of a first pipeline, a third port of the first three-way valve being communicated to an end of a second pipeline, and the vacuum pump being disposed on the second pipeline;

a first port of the second three-way valve being communicated to an other end of the first pipeline, and a second port of the second three-way valve being communicated to an end of a third pipeline, a third port of the second three-way valve being communicated to an end of a fourth pipeline (38), and an other end of the fourth pipeline being connected with the radon concentration measure instrument, and the radon collection box being arranged on the fourth pipeline;

a first port of the third three-way valve being communicated to an other end of the second pipeline, and a second port of the third three-way valve being communicated to an other end of the third pipeline, and a third port of the third three-way valve being communicated to a fifth pipeline.

In some embodiments, a first valve is installed on the air outlet pipeline, a second valve being installed on the second pipeline, a third valve being disposed on the third pipeline, and a fourth valve and a fifth valve being disposed on the fourth pipeline, and the fourth valve being disposed between the radon collection box and an end of the fourth pipeline, the fifth valve being disposed between the radon collection box and an other end of the fourth pipeline, and a sixth valve being disposed on the fifth pipeline.

In some embodiments, the device also includes a fourth three-way valve, a first port of the fourth three-way valve being communicated to an other end of the fourth pipeline, a second port of the fourth three-way valve being communicated to an inlet of the radon concentration measure instrument, and a third port of the fourth three-way valve being communicated to an outlet of the radon concentration measure instrument through a sixth pipeline, and the sixth pipeline being provided with a drying box filled with desiccant.

On the other aspect, the disclosure also provides a method for measuring radon release amount during rock shearing damage process. The method is performed on the basis of the above devices, and the method comprising:

placing the rock sample in the shear experiment instrument;

vacuumizing the sealed chamber with the vacuum pump;

completing a shearing process of the rock sample by the shear experiment instrument;

collecting radon in the rock sample into the radon collection box; and

measuring the concentration of the radon collected in the radon collection box with the radon concentration measure instrument.

The disclosure provides a device and method for measuring radon release amount during rock shearing damage process, the sealed chamber where the rock sample is installed is vacuumed in the first place, and then the radon released during rock sample shearing damage process is all collected into the radon collection box, and then the concentration of the radon collected in the radon collection box is measured with a radon concentration measure instrument, so that the purity of the radon collected in the radon collection box can be ensured, and thus the accuracy of the concentration of radon measured by the radon concentration measure instrument can be ensured, and the device and method have good practicability.

### BRIEF DESCRIPTION OF THE DRAWINGS

In order to more clearly explain the technical solutions in the embodiments of the disclosure, the drawings used in the description of the embodiments will be briefly introduced in the following. Apparently, the drawings in the following description only show some embodiments of the disclosure. For those of ordinary skill in the art, other drawings can be obtained based on these drawings without creative work.

FIG. 1 is a schematic diagram showing the structure of a device for measuring radon release amount during rock shearing damage process of an embodiment;

FIG. 2 is a schematic flow chart of a method for measuring radon release amount during rock shearing damage process according to an embodiment.

### DETAILED DESCRIPTION OF THE INVENTION

The technical solutions in the embodiments of the disclosure will be clearly and completely described below in conjunction with the accompanying drawings in the embodiments of the disclosure. Obviously, the described embodiments are only parts of the embodiments of the disclosure, rather than all the embodiments. Based on the embodiments of the disclosure, all other embodiments obtained by those of ordinary skill in the art without creative work shall fall within the protection scope of the disclosure.

First of all, in one aspect of the disclosure, it is provided a device for measuring radon release amount during rock shearing damage process.

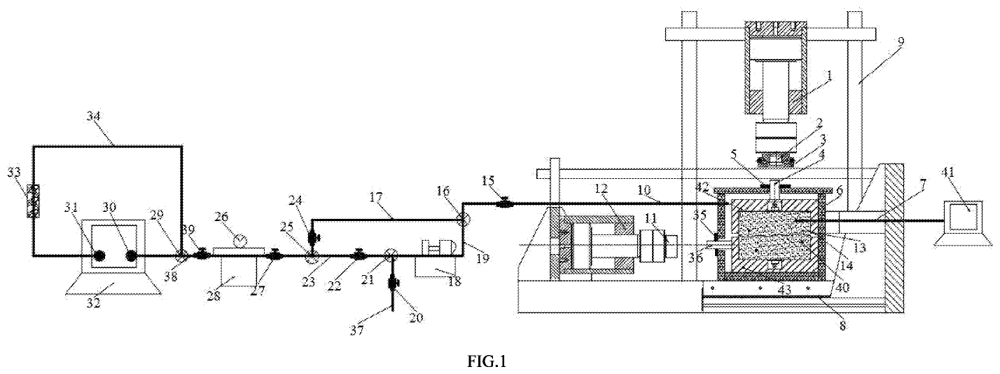

FIG. 1 is a schematic diagram showing a structure of a device for measuring radon release amount during rock shearing damage process according to one or more embodiments. With reference to FIG. 1, the device includes a shear experiment instrument, an acoustic emission sensor 40, an acoustic emission processor 41, a vacuum pump 18, a radon collection box 28 and a radon concentration measure instrument 32.

With reference to FIG. 1, the shear experiment instrument includes a normal loading mechanism, a tangential loading mechanism, a sealed chamber 6 and a shear box 14 for placing a rock sample 13, and the shear box 14 is disposed in the sealed chamber 6. The normal loading mechanism and the tangential loading mechanism are disposed on the outside of the sealed chamber 6. An output end of the normal loading mechanism is movable back and forth in the vertical direction, and the output end of the normal loading mechanism can pass through the sealed chamber 6 and the output end of the normal loading mechanism acts on the top of the shear box 14. An output end of the tangential loading mechanism is movable back and forth in the horizontal direction, and the output end of the tangential loading mechanism can pass through a horizontal side of the sealed chamber 6 and the output end of the tangential loading mechanism acts on a horizontal side of the shear box 14. A shear experiment can be performed on the rock sample 13 placed in the shear box 14 by operating the normal loading mechanism and the tangential loading mechanism.

Referring to FIG. 1, in some embodiments, the top of the sealed chamber 6 has an opening, and the top of the sealed chamber 6 is provided with a detachable cover plate 42 which can seal the opening. When measuring, the cover plate 42 is removed from the sealed chamber 6, and the shear box 14 carrying the rock sample 13 is placed in the sealed chamber 6, and then the cover plate 42 is installed on the opening of the top of the sealed chamber 6 in a sealed way, so that the rock sample 13 is in a sealed environment.

In some embodiments, the cover plate 42 can be installed on the top of the sealed chamber 6 by means of bolts, and a sealing element such as a sealing ring can be provided for them to improve the sealing effect of the sealed chamber 6.

Referring to FIG. 1, in some embodiments, the cover plate 42 is formed with a first through hole, and a first sealing gasket 5 is fixedly provided on the outer side of the cover plate 42, the first sealing gasket 5 covers the first through hole. The normal loading mechanism includes a normal cylinder 1 and a normal loading head 2. The normal cylinder 1 is fixedly arranged right above the cover plate 42, and a telescopic end of the normal cylinder 1 can be extendable and retractable downward in a vertical direction, an end of the normal loading head 2 is fixedly connected to the telescopic end of the normal cylinder 1, and an other end of the normal loading head 2 sequentially passes through the middle of the first sealing gasket 5 and the first through hole in the middle of the cover plate 42 and the other end of the normal loading head 2 acts on the top of the shear box 14.

With reference to FIG. 1, in some embodiments, the other end of the normal loading head 2 is detachably connected with a normal bearing head 4 through a spherical hinge fixing shaft 3, and the normal bearing head 4 sequentially passes through the middle of the first sealing gasket 5 and the first through hole in the middle of the cover plate 42 and the normal bearing head 4 acts on the top of the shear box 14 to apply a normal force to the shear box 14.

In some embodiments, the first sealing gasket 5 can also be arranged in the first through hole, and the sealing effect can also be achieved.

Referring to FIG. 1, in some embodiments, a horizontal side of the sealed chamber 6 is formed with a second through hole, a second sealing gasket 35 is fixedly provided on the outer side of the sealed chamber 6, the second sealing gasket 35 covers the second through hole. The tangential loading mechanism includes a tangential cylinder 12 and a tangential loading head 11. The tangential cylinder 12 is fixedly disposed on an outer side of the horizontal side of the sealed chamber 6, and a telescopic end of the tangential cylinder 12 can be extendable and retractable in the horizontal direction. An end of the loading head 11 is fixedly connected to the telescopic end of the tangential cylinder 12, and an other end of the tangential loading head 11 sequentially passes through the middle of the second sealing gasket 35 and the second through hole on the horizontal side of the sealed chamber 6, and the other end of the tangential loading head 11 acts on the horizontal side of the shear box 14.

Furthermore, referring to FIG. 1, in some embodiments, the other end of the tangential loading head 11 is detachably connected to a tangential bearing head 36, and the tangential bearing head 36 sequentially passes through the middle of the second sealing gasket 35 and the first through hole in the middle of the cover plate 42 and the tangential bearing head 36 acts on the side of the shear box 14 to apply a tangential force on the shear box 14.

In some embodiments, the second sealing gasket 35 can also be arranged in the second through hole, and the sealing effect can also be achieved.

In some embodiments, the normal bearing head 4 and the tangential bearing head 36 are both movably arranged in the through hole. When the interior of the sealed chamber 6 is kept in a vacuum state, a vacuum environment can be guaranteed inside the sealing chamber 6 during a shear damage experiment.

With reference to FIG. 1, in some embodiments, the shear box 14 includes a first box body 42 and a second box body 43. The first box body 42 is arranged above the second box body 43. The opposing end faces of the first box body 42 and the second box body 43 are both arranged with an opening. An end of the first box body 42 away from the tangential loading mechanism is fixedly provided with a counterforce frame 7, and the bottom of the second box body 43 is arranged on a roller bead row 8.

Furthermore, in some embodiments, the shear sample 13 is placed in the shear box 14. An other end of the normal bearing head 4 acts on the top of the first box body 42; the other end of the tangential loading head 11 acts on a side of the second box body 43 to generate a shearing force on the shear sample 13; the counterforce frame 7 can make the first box body 42 move in the tangential direction, and the second box body 43 can generate rolling friction on the roller bead row 8, in order to serve the shear test of shear sample 13.

Referring to FIG. 1, the shear experiment instrument of the embodiment may further include a framework 9. A fixed end of the normal cylinder 1, a fixed end of the tangential cylinder 12, the counterforce frame 7 and the roller bead row 8 are all fixedly installed on the framework 9.

Referring to FIG. 1, in some embodiments, a plurality of acoustic emission sensors 40 are arranged on a surface of the rock sample 13 at intervals, and the plurality of acoustic emission sensors 40 are all connected to an acoustic emission processor 41. The acoustic emission processor 41 can capture an acoustic emission signal from the acoustic emission sensors 40. The acoustic emission signal can reflect an expansion and development of cracks inside the rock sample during the loading process of the rock sample.

in some embodiments, the plurality of acoustic emission sensors 40 may be arranged on the surface of the shear sample 13 through coupling agent.

Referring to FIG. 1, in some embodiments, the vacuum pump 18 communicates with the interior of the sealed chamber 6 to pump air from the sealed chamber 6 so that the interior of the sealed chamber 6 is in a vacuum environment; the radon collection box 28 communicates with the interior of the sealed chamber 6 to collect radon generated during shear damage of the rock sample 13 into the radon collection box 28, and the radon concentration measure instrument 32 communicates to the radon collection box 28, and the radon concentration measure instrument 32 can be used to measure a concentration of radon collected in the radon collection box 28.

Specifically, with reference to FIG. 1, in some embodiments, the device includes a first three-way valve 16, a second three-way valve 25, and a third three-way valve 21, wherein the sealed chamber 6 is provided with a vent hole. The vent hole and a first port of the first three-way valve 16 are communicated through an air outlet pipeline 10, a second port of the first three-way valve 16 is communicated to an end of a first pipeline 17, and a third port of the first three-way valve 16 is communicated to an end of a second pipeline 19; the vacuum pump 18 is arranged on the second pipeline 19 to realize the communication between the vacuum pump 18 and the sealed chamber 6; and a first port of the second three-way valve 25 communicates with an other end of the first pipeline 17, and a second port of the third three-way valve 25 communicates with an end of a third pipeline 23, and a third port of the third three-way valve 25 communicates with an end of a fourth pipeline 38; an other end of the fourth pipeline 38 is connected to the radon concentration measure instrument 32, and the radon collection box 28 is arranged on the fourth pipeline 38 to realize the internal communication between the radon collection box 28 and the sealed chamber 6 and the communication between the radon concentration measure instrument 32 and the radon collection box 28. Furthermore, a first port of the third three-way valve 21 communicates with an other end of the second pipeline 19, a second port of the third three-way valve 21 communicates with an other end of the third pipeline 23, and a third port of the third three-way valve 21 communicates with the fifth pipeline 37.

Furthermore, referring to FIG. 1, in some embodiments, a first valve 15 is installed on the air outlet pipeline 10, a second valve 24 is installed on the second pipeline 17, a third valve 22 is provided on the third pipeline 23, and a fourth pipeline 38 is provided with a fourth valve 27 and a fifth valve 39, the fourth valve 27 is provided between the radon collection box 28 and an end of the fourth pipeline 38, the fifth valve 39 is provided between the radon collection box 28 and the other end of the fourth pipeline 38, and a sixth valve 20 is provided on the fifth pipeline 37, for on-off control of the corresponding pipe.

Furthermore, referring to FIG. 1, in some embodiments, a pressure meter 26 is also installed on the radon collection box 28 to determine the pressure of the radon in the radon collection box 28 to prevent accidents.

Referring to FIG. 1, in some embodiment, the device may also include a fourth three-way valve 29. A first port of the fourth three-way valve 29 communicates with an other end of the fourth pipeline 38, and a second port of the fourth three-way valve 29 communicates to the inlet of the radon concentration measure instrument 32, and a third port of the fourth three-way valve 29 communicates to the outlet of the radon concentration measure instrument 32 through a sixth pipeline 34. The sixth pipeline 34 is provided with a drying box 33 filled with desiccant. In this way, the radon concentration measure instrument 32 can be used for cyclic measurement to improve the accuracy of concentration measurement.

Based on the above device, an embodiment also provides a method for measuring radon release amount during rock shearing damage process.

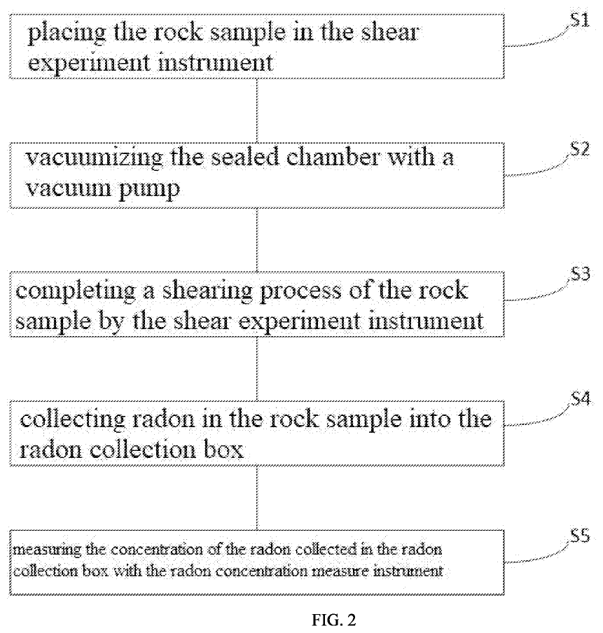

FIG. 2 is a schematic flow chart of a method for measuring radon release amount during rock shearing damage process according to the embodiment. With reference to FIG. 2, the method includes:

S1: placing a rock sample in the shear experiment instrument, including:

removing the cover plate 42 from the sealed chamber 6, and placing the shear box 14 carrying the rock sample 13 in the sealed chamber 6, and then installing the cover plate 42 on the opening of the top of the sealed chamber 6 in a sealed way.

The method may further include S2: vacuumizing the sealed chamber with a vacuum pump, including:

firstly, an experimental temperature is set to be constant, for example 23.5° C.; the vacuum pump 18 is turned on to vacuumize the entire device including the sealed chamber 6; the sixth valve 20 is opened, and the first valve 15, the third valve 22, the second valve 24, the fourth valve 27 and the fifth valve 39 are all opened; after the vacuum is completed, the sixth valve 20 and the fifth valve 39 are closed; the experimental device are kept as it is; if the pressure does not change for a period of time, the experimental device is regarded as being airtight; if there is a change in pressure, the air tightness is checked until there is no gas leakage.

The method may further include S3: completing a shearing process of the rock sample by the shear experiment instrument, including:

the first valve 15 is closed; the normal cylinder 1 is started to perform loading experiment, and at the same time, the acoustic emission sensors are started to collect acoustic emission events during the loading process; the normal cylinder 1 applies a normal load to the shear sample through the normal bearing head 4; after a normal pressure is stabilized, the tangential cylinder 12 is started to perform shear loading, wherein the tangential cylinder 12 applies a transverse load to the shear sample through the tangential bearing head 36. The test data are recorded.

The method may further include S4: collecting radon in the rock sample into the radon collection box, specifically including:

after the shear experiment is completed, the first valve 15, the second valve 24, and the fourth valve 27 are opened; under the action of the pressure difference, radon quickly enters the radon collection box 28; the vacuum pump 18 is turned on, and the sixth valve 20 and the second valve 24 are closed, the third valve 22 is opened to pump the gas in the pipelines into the radon collection box 28, so as to collect the radon in the pipelines into the radon collection box 28.

The method may further include S5: measuring the concentration of the radon collected in the radon collection box with the radon concentration measure instrument, specifically including:

after the radon in the pipelines is collected into the radon collection box 28, the fourth valve 27 is closed, and the fifth valve 39 and the radon concentration measure instrument 32 are opened; the concentration of accumulated radon are measured by the radon concentration measure instrument.

In summary, embodiments of the disclosure provide a device and method for measuring radon release amount during the rock shear damage process, in which the sealed chamber where the rock sample placed is vacuumed in the first place, and then the radon released during rock sample shearing damage process is all collected into the radon collection box, and then the concentration of the radon collected in the radon collection box is measured with a radon concentration measure instrument, so that the purity of the radon collected in the radon collection box can be ensured, and thus the accuracy of the concentration of radon measured by the radon concentration measure instrument can be ensured, and the device and method have good practicability.

The forgoing embodiments are preferred embodiments of the disclosure, which are simply used to facilitate the description of the disclosure, and are not intended to limit the disclosure in any form. Any equivalent embodiments made by those skilled in the art without departing from the scope of technical features of the disclosure by partially changing or modifying the disclosed technical content disclosed in the disclosure are still fall within the scope of the technical features of the disclosure.

What is claimed is:

1. A device for measuring radon release amount during rock shearing damage process, comprising:
a shear experiment instrument, the shear experiment instrument including a normal loading mechanism, a tangential loading mechanism, a sealed chamber (6) and a shear box (14) for placing a rock sample (13), the shear box (14) being disposed in the sealed chamber (6), the normal loading mechanism and the tangential loading mechanism being disposed on the outside of the sealed chamber (6), and an output end of the normal loading mechanism being movable back and forth along a vertical direction, the output end of the normal loading mechanism being capable of passing through the sealed chamber (6) and the output end of the normal loading mechanism acting on a top of the shear box (14), and an output end of the tangential loading mechanism being movable back and forth along a horizontal direction, the output end of the tangential loading mechanism being capable of passing through a horizontal side of the sealed chamber (6), and the output end of the tangential loading mechanism acting on the horizontal side of the shear box (14);
an acoustic emission sensor (40) and an acoustic emission processor (41), a plurality of the acoustic emission sensors (40) being disposed on a surface of the rock sample (13) at intervals, and a plurality of the acoustic emission sensors (40) being connected to the acoustic emission processor (41);
a vacuum pump (18), the vacuum pump (18) being communicated with an interior of the sealed chamber (6);
a radon collection box (28), the radon collection box (28) being communicated with the interior of the sealed chamber (6);
a radon concentration measure instrument (32), the radon concentration measure instrument (32) being communicated with the radon collection box (28);
the device including a first three-way valve (16), a second three-way valve (25) and a third three-way valve (21), wherein:
the sealed chamber (6) being provided with a vent hole, the vent hole being communicated to a first port of the first three-way valve (16) through an air outlet pipeline (10), and a second port of the first three-way valve (16) being communicated to an end of a first pipeline (17), a third port of the first three-way valve (16) being communicated to an end of a second pipeline (19), and the vacuum pump (18) being disposed on the second pipeline (19);
a first port of the second three-way valve (25) being communicated to an other end of the first pipeline (17), and a second port of the second three-way valve (25) being communicated to an end of a third pipeline (23), a third port of the second three-way valve (25) being communicated to an end of a fourth pipeline (38), and an other end of the fourth pipeline (38) being connected with the radon concentration measure instrument (32), and the radon collection box (28) being arranged on the fourth pipeline (38);
a first port of the third three-way valve (21) being communicated to an other end of the second pipeline (19), and a second port of the third three-way valve (21) being communicated to an other end of the third pipeline (23), and a third port of the third three-way valve (21) being communicated to a fifth pipeline (37);
a first valve (15) being installed on the air outlet pipeline (10), a second valve (24) being installed on the second pipeline (17), a third valve (22) being disposed on the third pipeline (23), and a fourth valve (27) and a fifth valve (39) being disposed on the fourth pipeline (38), and the fourth valve (27) being disposed between the radon collection box (28) and an end of the fourth pipeline (38), the fifth valve (39) being disposed between the radon collection box (28) and an other end of the fourth pipeline (38), and a sixth valve (20) being disposed on the fifth pipeline (37); and
the device also including a fourth three-way valve (29), a first port of the fourth three-way valve (29) being communicated to an other end of the fourth pipeline (38), a second port of the fourth three-way valve (29) being communicated to an inlet of the radon concentration measure instrument (32), and a third port of the fourth three-way valve (29) being communicated to an outlet of the radon concentration measure instrument (32) through a sixth pipeline (34), and the sixth pipeline (34) being provided with a drying box (33) filled with desiccant.

2. The device for measuring radon release amount during rock shearing damage process according to claim 1, wherein a top of the sealed chamber (6) is arranged with an opening, and the top of the sealed chamber (6) is provided with a detachable cover plate (42), and the opening is sealed by the cover plate (42).

3. The device for measuring radon release amount during rock shearing damage process according to claim 2, wherein the cover plate (42) is formed with a first through hole, and a first sealing gasket (5) is fixedly arranged on an outer side of the cover plate (42), and the first through hole is covered by the first sealing gasket (5); and
the normal loading mechanism includes a normal cylinder (1) and a normal loading head (2), the normal cylinder (1) being fixedly disposed right above the cover plate (42), a telescopic end of the normal cylinder (1) being extendable and retractable downward in a vertical direction, an end of the normal loading head (2) being fixedly connected to the telescopic end of the normal cylinder (1), and an other end of the normal loading head (2) sequentially passing through the middle of the first sealing gasket (5) and the first through hole in the middle of the cover plate (42), and an other end of the normal loading head (2) acting on the top of the shear box (14).

4. The device for measuring radon release amount during rock shearing damage process according to claim 1, wherein the horizontal side of the sealed chamber (6) is formed with a second through hole, and a second sealing gasket (35) is fixedly provided on the outer side of the sealed chamber (6), and the second through hole is covered by the second sealing gasket (35); and
the tangential loading mechanism includes a tangential cylinder (12) and a tangential loading head (11); the tangential cylinder (12) is fixedly disposed on an outer side of the horizontal side of the sealed chamber (6), a telescopic end of the tangential cylinder (12) being extendable and retractable in a horizontal direction, an end of the tangential loading head (11) being fixedly connected to the telescopic end of the tangential cylinder (12), an other end of the tangential loading head (11) sequentially passing through the middle of the second sealing gasket (35) and the second through hole on the horizontal side of the sealed chamber (6), and an other end of the tangential loading head (11) acting on a horizontal side of the shear box (14).

5. The device for measuring radon release amount during rock shearing damage process according to claim 1, wherein the shear box (14) includes a first box body (42) and a second box body (43), and the first box body (42) is arranged above the second box body (43); the opposing end faces of the first box body (42) and the second box body (43) are both arranged with an opening; an end of the first box body (42) away from the tangential loading mechanism is fixedly provided with a counterforce frame (7); and the bottom of the second box body (43) is arranged on a roller bead row (8).

6. The device for measuring radon release amount during rock shearing damage process according to claim 1, wherein the plurality of the acoustic emission sensors (40) are arranged on the surface of the shear sample (13) by a coupling agent.

7. A method for measuring radon release amount during rock shearing damage process, wherein the method is preformed on the basis of the device according to claim 1, the method comprising:
placing the rock sample (13) in the shear experiment instrument;
vacuumizing the sealed chamber (6) with the vacuum pump (18);
completing a shearing process of the rock sample (13) by the shear experiment instrument;
collecting radon in the rock sample (13) into the radon collection box (28); and
measuring the concentration of the radon collected in the radon collection box (28) with the radon concentration measure instrument (32).

8. A method for measuring radon release amount during rock shearing damage process, wherein the method is preformed on the basis of the device according to claim 2, the method comprising:
placing the rock sample (13) in the shear experiment instrument;
vacuumizing the sealed chamber (6) with the vacuum pump (18);
completing a shearing process of the rock sample (13) by the shear experiment instrument;
collecting radon in the rock sample (13) into the radon collection box (28); and
measuring the concentration of the radon collected in the radon collection box (28) with the radon concentration measure instrument (32).
